(19) United States
(12) Patent Application Publication
(10) Pub. No.: US 2021/0052025 A1
(43) Pub. Date: Feb. 25, 2021

(54) INFUSION FRIENDLY SWEATER DONNABLE WITH MEDICAL ATTACHMENTS

(71) Applicant: Aleah Colon-Alfonso, Sarasota, FL (US)

(72) Inventor: Aleah Colon-Alfonso, Sarasota, FL (US)

(21) Appl. No.: 16/544,370

(22) Filed: Aug. 19, 2019

(51) Int. Cl. A41D 13/12; A41D 1/04; A61M 5/14; A41D 27/20; A41D 27/28; A41D 27/10

(52) U.S. Cl. CPC A41D 13/1245; A41D 1/04; A61M 5/14; A61M 2025/0206; A41D 27/28; A41D 27/10; A41D 27/20

(57) ABSTRACT

A comfort, infusion-friendly, sweater supplements existing attire of a wearer and accommodates pre-existing medical attachments. The sweater includes a first arm portion with top and bottom plackets extending lengthwise from a collar portion to a wrist portion, plackets having fasteners and mating fasteners. The sweater has a first front portion connected to the first arm portion, a second arm portion, a second front portion and a back. A collar pull tab, having a pull fastener, extends outward near the collar. A mating fastener is on the collar portion or the back portion. Engaging the first arm fasteners and mating fasteners dons the sweater over the existing attire of the wearer, without disruption the medical connections. Based on the plackets and pull tab(s), a wearer can single-handedly don and remove the sweater, adding self-sufficient comfort with a medical procedure.

### COPYRIGHT NOTICE

A portion of the disclosure of this patent document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### RELATED APPLICATIONS

There are no related applications.

### FIELD OF INVENTION

The disclosed technology relates generally to a patient's garment and more specifically to a supplemental garment for donning without disrupting medical attachments.

### BACKGROUND

Patients connected to medical devices can find themselves subjected to temperature fluctuations while undergoing lengthy routine procedures. For examples, procedures may be dialysis, chemotherapy, radiation, or the like. A patient is typically hooked-up to the medical device via an arm or thorax.

With these medical attachments, patients have limited options to help better regulate their own temperature. One approach is for the patient to dress for cooler temperatures and supplement with a blanket that can be laid over the patient. Because the patient is physically connected with medical attachments, the patient cannot pull a normal sweater over themselves. The same problem occurs with sleeves and medical attachments getting caught in the sleeves.

One existing solution is the Mighty Wrap available from Mighty Well. This product is a very large and roomy open-front sweater with large arm sockets. This solution suffers from the same complications as other prior art solutions, the wearer must either snake medical attachment connections through the front opening of the wrap or don the product prior to medical attachment and keep the wrap on during the whole procedure. Additionally, this solution risks dislocation or disruption of the IV or other attachment.

There are several known hospital gown solutions. These all suffer from the same deficiency of being directed to a primary gown to cover a patient, not providing supplementation for temperature regulation. For a hospital gown, the patient removes all underlying clothes and the gown is about access to the patient's body, not accounting for comfort and temperature regulation.

U.S. Pat. No. 4,570,268 (“the '268 Patent”) is a three-piece patient garment with various fasteners extending down the arms and across the front. The '268 Patent hospital garment is the sole article of clothing worn by the patient, and it includes short sleeves, its three-piece construction allows for medical-access to the patient. The access is for in-hospital medical procedures, such as allowing for x-rays, thorax and abdomen examinations, even a nursing mother access to breastfeed. Nothing in the '268 Patent provides for temperature regulation or supporting/comforting a wearer while undergoing medical procedures. Moreover, the '268 Patent gown is donned by the patient prior to undertaking any medical procedures, with the various fasteners defining later medical doctor access.

U.S. Publication No. 2013/0276202 (“the '202 Pub.”) teaches a front-opening hospital gown with overlapping front panels. The '202 Pub. hospital gown teaches fasteners along a portion of the sleeves, where opening the fasteners allows medical access to the arms. This '202 Pub. gown is a typical high-use medical gown designed as the primary patient covering garment and able to be subjected to a large number of high intensity washings. This gown does not seek providing comfort to the patient. The '202 Pub. gown is very similar to the '268 Patent gown in allowing ease of doctor access to the patient wearing only the gown itself.

U.S. Publication No. 2015/0157069 (“the '069 Pub.”) teaches a patient gown similar to the '202 publication and the '268 Patent. This gown is a three panel hospital gown, donned on the patient without any additional clothing, with the fastener locations granting access to the patient. Because the '069 Pub. gown is a primary clothing article, the fasteners are made with higher stability fasteners to survive the repeated high temperature washings and the gown is designed to include privacy for the otherwise-naked patient.

These existing solutions all fail to provide donning and removal options for the patient while attached to one or more medical devices, in conjunction with allowing the patient to wear his or her own clothing. The wrap solutions bunch or otherwise constrict existing medical connections. The hospital gown solutions are not meant for comfort to the patient based on being a hospital gown, are not directed to donning and removal options during treatment because the gown is worn prior to commencement of any procedure, and these existing solutions including design complications inhibiting a wearer to don and remove the clothing without additional assistance.

As such, there exists a need for garment, an infusion friendly sweater, that accommodates medical attachments to the wearer and allows for quick and easy one-handed donning and removal of the infusion friendly sweater due to temperature fluctuations.

### BRIEF DESCRIPTION

An infusion friendly comfort sweater supplements existing attire of a wearer already connected to and having pre-existing medical attachments. The infusion friendly sweater includes arm portions, front portions, a back portion and a collar portion.

A first arm portion has a first arm top placket and a first arm bottom placket. These plackets extend lengthwise from the collar portion to a wrist portion of the first arm portion. The top placket includes a plurality of first arm fasteners and the bottom placket has a plurality of first arm mating fasteners for engagement with the plurality of first arm fasteners.

A first front portion is connected to the first arm portion. A second arm portion extends outward from the collar portion to a second wrist portion of the second arm portion. The second arm portion may also include a plurality of plackets with fasteners and mating fasteners extending lengthwise. The back portion connects to the collar portion, the arm portions, and the front portions.

The sweater further includes a collar pull tab extending outward from either the first front portion or the second front portion. The collar pull tab has a pull fastener thereon. A pull mating fastener is disposed on the collar portion and/or the back portion.

Thereby, the infusion friendly sweater can be donned by the wearer to supplement existing attire and without disruption of existing medical attachments. Donning the sweater includes wrapping the first arm portion around the wearer's arm and then engaging the first arm fasteners with the first arm mating fasteners. Moreover, engaging the pull fastener with the pull mating fastener secures the sweater around the wearer. The spaced-apart nature of the fasteners creates apertures through which the medical attachments can pass while the placket is in a closed position.

Varying embodiments provide for different donning techniques as well as for securing the sweater around the wearer. For example, the front portions may include plackets with fasteners and mating fasteners to secure the front portions around the wearer. In another embodiment, the second arm can also include plackets with fasteners and mating fasteners to be wrapped around the arm of the wearer.

Different embodiments can include the collar pull tab and/or a wrist pull tab. The pull tabs, extending outward from the sweater, allow the wearer to secure the sweater by themselves, using a free hand. For example, the collar pull tab allows the wearer to secure the sweater in place if one arm cannot be moved because it is connected to a medical attachment. Thus, using the pull(s), the wearer can don and remove the sweater using a single free hand.

The fasteners can be disposed in any suitable spacing along the plackets. Additionally, the fasteners can be any device or devices for donning the sweater, such as but not limited to magnets, buttons, snaps, hook-and-loop connectors, etc., or a combination thereof.

The medical attachments can be any suitable attachment to the person. Examples can include intravenous (IV) lines, peripherally inserted central catheters, a dialysis connection, etc.

Therein, the infusion friendly sweater, via the multiple plackets and the pull tab(s), allows for donning over pre-existing attire by a wearer having pre-existing medical attachments. The infusion friendly sweater is worn over the wearer's pre-existing clothing and can be made of soft, comfortable material to help regulate body temperature. The infusion friendly comfort sweater allows a wearer undergoing a medical procedure, such as an infusion, to self-regulate and better adjust to temperature fluctuations, by donning and removing the sweater without adversely impacting the connected medical attachments.

### BRIEF DESCRIPTION OF THE DRAWINGS

FIG. 1 illustrates a front view of an infusion friendly sweater with the fasteners engaged;

FIG. 2 illustrates a front view of the infusion friendly sweater with the fasteners disengaged, the sweater fully open prior to donning;

FIG. 3 illustrates a partial view of the sweater being donned around a wearer's arm;

FIG. 4 illustrates a partial view of the sweater being donned around a wearer having a medical attachment;

FIG. 5 illustrates a partial view of the donned sweater with the wearer having a medical attachment;

FIG. 6 illustrates a partial front view of the collar tab of the sweater;

FIG. 7 illustrates a partial back view of the collar portion of the sweater;

FIG. 8 illustrates a partial front view of the collar tab engagement on the sweater;

FIGS. 9a-9b illustrate partial back views of the collar tab engagement on the sweater; and

FIG. 10 illustrates a partial top view of the sweater donned without disruption of a medical attachment.

A better understanding of the disclosed technology will be obtained from the following detailed description of the preferred embodiments taken in conjunction with the drawings and the attached claims.

### DETAILED DESCRIPTION

As described herein, a wearer undergoing medical treatment can better regulate body temperature using the infusion friendly sweater. The infusion friendly sweater, through its unique construction allows for donning and removal without disruption of existing medical attachments, e.g. catheter, IV line(s), etc. The infusion friendly sweater further allows donning and removal individually by the wearer using a single-hand where the other hand is rendered immobile from the medical attachment(s).

FIG. 1 illustrates a front view of one embodiment of an infusion friendly sweater 100 in a closed position. The sweater 100 includes a first arm portion 102, a second arm portion 104, a first front portion 106, a second front portion 108, a back portion 110, and a collar portion 112. The arm portions 102, 104 include plackets 114, 116. The first front portion 106 include a placket 118. The second front portion 108 also includes a placket, not visible as being occluded in FIG. 1 by the placket 118.

A placket is an opening on a piece of clothing allowing for donning and removal of the clothing item. The placket can include attachments for opening/closing the placket, for example buttons and button-holes or snaps found on plackets on a blouse or dress shirt. Plackets can be functional and/or decorative.

The infusion friendly sweater 100, by having multiple plackets at the arms and down the front, allows for donning and removal without disruption of medical attachments connected to a wearer.

The infusion friendly sweater 100 includes a collar pull tab 120, a first wrist tab 122 and a second wrist tab 124. The first wrist tab 122 is disposed at a first wrist portion 126 of the first arm portion 102, the second wrist tab 124 is disposed at a second wrist portion 128 of the second arm portion 104.

While not visible in FIG. 1, the plackets 114, 116, 118 include fasteners and mating fasteners. As used herein, fasteners and mating fasteners provide for interconnection to secure the placket closed. A fastener and mating fasteners are complimentary elements. Fasteners and mating fasteners can include magnets, buttons and button-holes, hook-and-loop fasteners, zippers, clasps, or any other suitable fastener, or a combination of different fasteners, as recognized by one skilled in the art.

The infusion friendly sweater 100 includes the collar pull tab 120 that additionally allows securing the sweater 100 around a wearer. The wrist pull tab 122 and/or 124 additionally secures the sweater 100 in place.

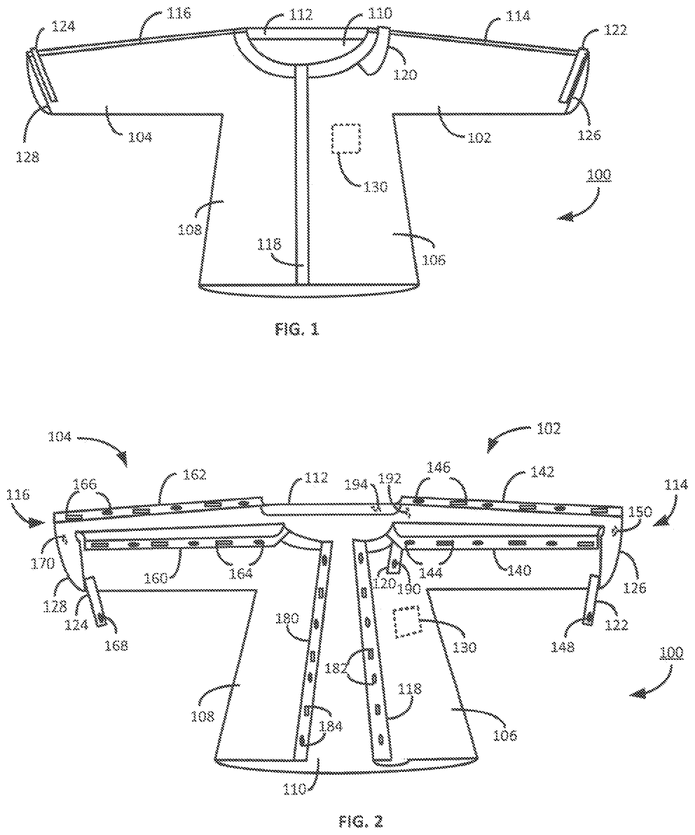

One embodiment may include one or more interior or hidden pockets, such as pocket 130. Additionally, the sweater 100 may include exterior pockets (not shown), including pockets for placing hands therein, or pocket(s) for carrying items. For example, the sweater 100 may allow an interior pocket 130 for carrying a medical device or accoutrements associated with the medical attachment. The pocket or pockets can also include heat packets or cool packets to further effectuate temperature adjustment of the wearer.

Where FIG. 1 illustrates the closed sweater 100, FIG. 2 illustrates the sweater 100 in an open position. In FIG. 2, the sweater 100 is in a position for being donned around a wearer having connected medical attachments.

FIG. 2 illustrates the sweater 100 with the first arm portion 102, second arm portion 104, first front portion 106, second front portion 108, back portion 110, and collar portion 112. When open, the first arm portion 102 includes a top placket 140 and a bottom placket 142. The top placket 140 includes a plurality of fasteners 144 with the bottom placket 142 including mating fasteners 146. Additionally, the wrist tab 122 includes a fastener 148 with a mating fastener 150 embedded in either arm portion 104 or the wrist portion 126.

In one embodiment, the second arm portion 104 includes similar construction with a top placket 160 and bottom placket 162. The top placket 160 includes fasteners 164 and the bottom placket 162 has mating fasteners 166. The wrist tab 124 includes a fastener 168. A corresponding mating fastener 170 is in either the arm portion 102 or the wrist portion 170.

In the front, the first front portion 106 has front placket 118 and the second front portion 108 has an interior placket 180. The front placket 118 has a plurality of spaced-apart fasteners 182 and the interior placket 180 has mating fasteners 184.

The collar pull tab 120 is shown in an open position, with fastener 190. A mating fastener 192 and/or 194 can be disposed on the back portion 110 or the collar portion 112 such that when attached it further secures the sweater 100 around the wearer. The collar pull tab 120 also has an extended length allowing for connecting the fastener 190 and mating fastener 192 or 194 using one hand in the event the other hand is immobile.

As noted above, the fasteners and mating fasteners on the plackets are spaced apart. The plackets allow for wrapping the sweater around the wearer and securing the sweater in place by connecting the fasteners and mating fasteners. The pull tab(s) secure(s) the sweater in place when the wearer has limited mobility. Additionally, the donning and removal of the sweater is over the wearer's existing clothing. The sweater is a supplemental garment allowing the wearer to keep on existing clothing and add/remove the sweater solely for comfort purposes.

The spaced apart nature of the fasteners accommodates pre-existing medical attachments. By having spacing between fasteners, the placket naturally creates an opening accommodating the attachments.

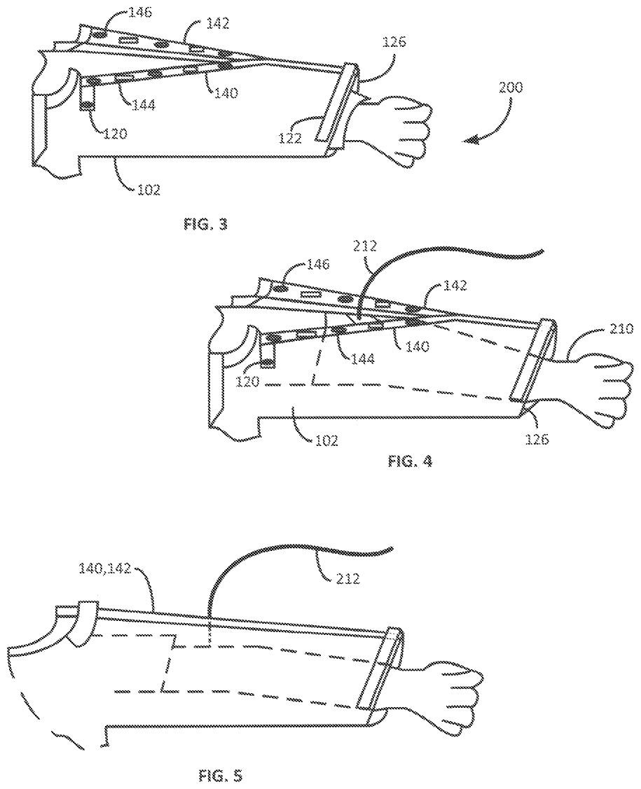

FIG. 3 illustrates a partial view of a wearer 200 donning the first arm portion 102 of the sweater 100. Here, the wearer 200 has on regular clothes such as a long-sleeved shirt. In FIG. 3, the arm is not connected to any medical attachments, but donning the sweater can include sliding the arm through the wrist portion 126 opening or wrapping the arm portion 102 around the wearer's arm and closing the fasteners 144 and mating fasteners 146 on the plackets 140, 142. Also visible, the wrist tab 122 is secured in place and the collar tab 120 is open.

FIG. 4 illustrates a wearer arm 210 with a medical attachment 212 connected thereto. As noted above, the medical attachment 212 can be any suitable device or devices connected to the wearer, such as a catheter, IV line, electrical monitoring connection(s), etc.

With prior solutions, as the wearer inserted his or her arm into the garment, the attachment 212 would be caught within an interior of the sleeve. Or, in the alternative, the wearer would have to remove the attachment 212, the arm slid through the arm socket, and then the attachment 212 re-attached.

By contrast, the wearer secures the arm portion 102 of the infusion friendly sweater around the wearer's arm 210 by selectively connecting fasteners 146 and mating fasteners 148 along the plackets 140, 142. For clarity purposes, FIG. 4 illustrates the fasteners and mating fasteners already engaged on the half of the arm portion 102 nearer the wrist portion 126.

As the plackets 140 and 142 are connected, FIG. 5 illustrates a front view of the infusion friendly sweater fully donned around the wearer's arm 210. As illustrated, the medical attachment 212 remains undisturbed, extending through an offset opening made possible by the spaced-apart fasteners and mating fasteners from the plackets 142, 144.

The infusion friendly sweater also includes a collar tab assisting with single-handed donning and removal. The wearer is typically connected to one or more machines. These medical treatments can take an extended period of time, where the wearer has limited mobility and can find themselves subject to temperature fluctuations. For example, during a chemotherapy treatment, the wearer sits for an extended time period with limited mobility because of an IV line.

These medical treatment sessions are often performed with minimal medical supervision, such as a nurse or assistant periodically checking in on the wearer. Thus, the infusion friendly sweater allows for donning the sweater without outside assistance. With the wearer having limited mobility, the collar pull tab allows the wearer to secure the sweater in place using a single free hand.

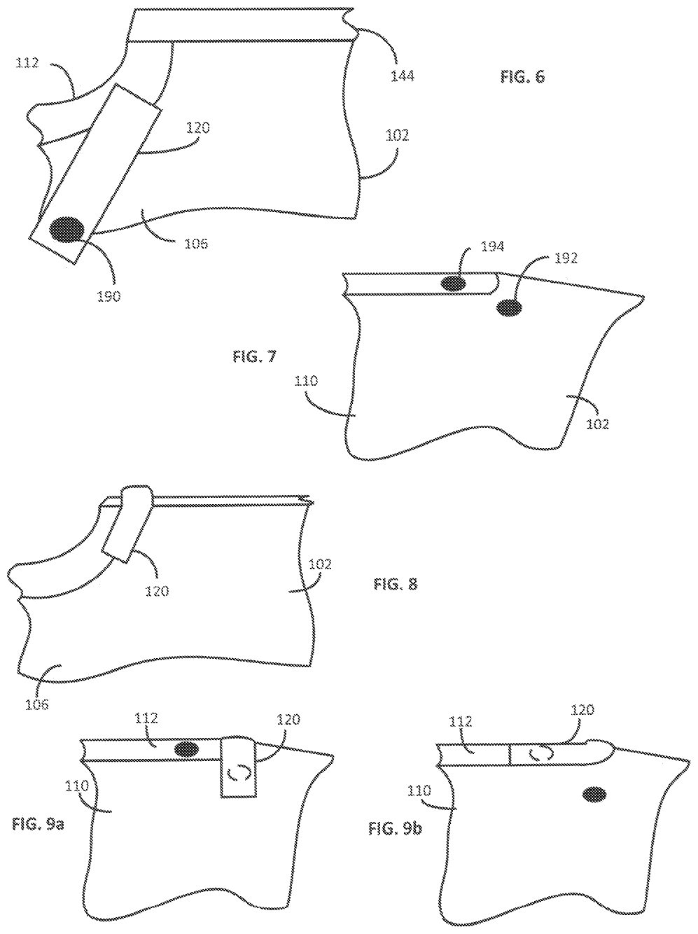

FIGS. 6-9b illustrate one embodiment of the collar pull tab. FIG. 6 is a partial view of the arm portion 102 with the collar pull tab 120 in an open position, e.g. not secured around the wearer. The fastener 190 is disposed on a distal end of the tab 120. The pull tab 120 is disposed, in this exemplary embodiment, connected to the front portion 106. It is recognized the pull tab 120 may be affixed to the arm top placket 144, the arm portion 102, or even the collar portion 112. The specific location of the tab 120 connected to the sweater can be dependent on the length of the tab itself.

Where FIG. 6 is a front view, FIG. 7 is a rear view of the infusion friendly sweater with the arm portion 102 and the back portion 110. Visible in FIG. 7 are two exemplary mating fasteners 192, 194. The mating fasteners 192, 194 are for receipt and mating with the fastener 190 of FIG. 6 when the tab is secured in place.

FIG. 7 illustrates the alternative embodiments of various mating fastener locations 192, 194. The mating fastener 220 can allow for more of an open front position of the sweater when secured and the mating fastener 222 can create a modified turtleneck structure. A mating fastener may also be disposed on the arm portion, for example if the wearer has limited mobility and unable to reach over up to the collar or back portion.

FIGS. 8-9b illustrate front and rear views in a closed position. FIG. 8 illustrates the front portion 106 with the collar pull tab 120 pulled up and around the arm portion 102. FIGS. 9a-b illustrate the rear view showing the pull tab secured in place. FIG. 9a uses the mating fastener on the back portion 110. FIG. 9b uses the mating fastener on the collar portion 112.

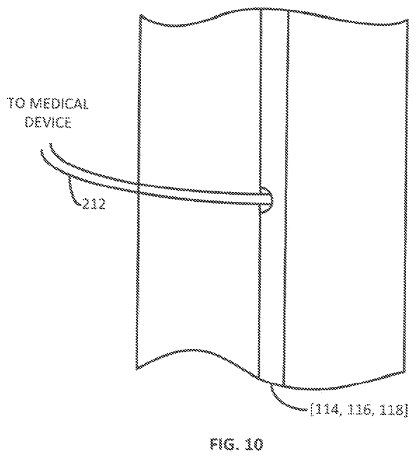

For further clarity, FIG. 10 illustrates a top view of a closed placket. The placket may be an arm placket 114 and 116. The placket can also be a front placket 118. FIG. 10 illustrates the connecting of the fasteners and mating fasteners does not adversely affect the medical attachment 212. Therefore, the wearer being already connected to medical device(s) through the attachment(s) 212 can readily don and remove the infusion friendly sweater.

The infusion friendly sweater may include varying embodiments offering varying degrees of options for the wearer. For example, FIGS. 1-2 illustrate one embodiment having multiple plackets allowing for fully wrapping the sweater around the wearer prior to closing the plackets.

In another embodiment, the sweater may have a single fastener/mating fastener placket on one arm, with the other arm being a standard closed channel found in a standard sweater. In this embodiment, the wearer has medical attachments to one arm, the other arm is free to slide through the wrist opening, leaving the arm with the open plackets to be wrapped around the wearer and the medical attachment.

As noted above, the front portion may close using plackets with fasteners and mating fasteners. But further embodiments may use other types of closures, such as extra material to fold over, string or ties to tie up the front for examples.

The infusion friendly sweater, not being a primary medical clothing item, e.g. a hospital gown, is not subject to manufacturing specifications directed to repeated high-temperature washing. The infusion friendly sweater can be made of any suitable material allowing for comfort to the patient, such as soft cotton, wool, synthetic materials, blends of different materials. The infusion friendly sweater can be made of a material designed to retain heat in addition to the comfort. For example different materials can promote heat retention, for example wool, thicker cotton, synthetic materials, etc. The sweater may also include inserts or inner layers of material to further promote temperature regulation.

In varying embodiments, the sweater may include designs on the material or attached to the material. For example, for a child sweater, the infusion friendly sweater may include pictures of animals. In another example, for a child the sweater may include hook-and-loop fasteners for attaching a stuff animal to the outside or an exterior pocket for holding the animal.

One embodiment may include pockets for holding additional equipment associated with the wearer's medical treatment. These pockets can also hold personal items. Pockets can be internal and/or external pockets, positioned to assist the wearer without interfering with its ease of donning and removal.

Further embodiments may include internal pockets or channels for circulating airflow. For example, various pockets can be located at different areas/regions of the sweater. In one example, pockets near the front portions can hold hot or cold packets to help temperature regulation at the wearer's torso. In another embodiment, pocket(s) can be within the arm portion for assisting with discomfort of the medical attachments, such as a pocket to hold a cold packet near the insertion point of the medical attachment.

The sweater may include pockets or channels connectable to an external air flow generator that sends cooled or heated air for internal circulation within the sweater. For example, an air circulation device can generate cooled or heated air, and upon insertion into an interior channel within the sweater, the cool or heated air can circulate to better comfort the wearer. The interior channel may include a channel formed by the closing of the plackets.

Moreover, as the infusion friendly sweater is a secondary or supplementary clothing article, the sweater can use larger dimensions for draping over the wearer. For example, the sweater can be designed with larger proportions from a standard sized sweater, allowing for roomier fitting and more draping over the wearer.

FIGS. 1 through 9b are conceptual illustrations allowing for an explanation of the present invention. Notably, the figures and examples above are not meant to limit the scope of the present invention to a single embodiment, as other embodiments are possible by way of interchange of some or all of the described or illustrated elements. Moreover, where certain elements of the present invention can be partially or fully implemented using known components, only those portions of such known components that are necessary for an understanding of the present invention are described, and detailed descriptions of other portions of such known components are omitted so as not to obscure the invention. In the present specification, an embodiment showing a singular component should not necessarily be limited to other embodiments including a plurality of the same component, and vice-versa, unless explicitly stated otherwise herein. Moreover, Applicant does not intend for any term in the specification or claims to be ascribed an uncommon or special meaning unless explicitly set forth as such. Further, the present invention encompasses present and future known equivalents to the known components referred to herein by way of illustration.

The foregoing description of the specific embodiments so fully reveals the general nature of the invention that others can, by applying knowledge within the skill of the relevant art(s) (including the contents of the documents cited and incorporated by reference herein), readily modify and/or adapt for various applications such specific embodiments, without undue experimentation, without departing from the general concept of the present invention. Such adaptations and modifications are therefore intended to be within the meaning and range of equivalents of the disclosed embodiments, based on the teaching and guidance presented herein.

1. An infusion friendly sweater_configured to supplement existing attire of a wearer and configured to accommodate pre-existing medical attachments connected to the wearer, the sweater comprising:
a first arm portion having a first arm top placket and a first arm bottom placket, both the first arm top placket and the first arm bottom placket extending lengthwise from a collar portion to a first wrist portion of the first arm portion, the first arm top placket including a plurality of first arm fasteners and the first arm bottom placket including a plurality of first arm mating fasteners for engagement with the plurality of first arm fasteners;
a first front portion connected to the first arm portion,
a second arm portion extending from the collar portion to a second wrist portion of the second arm portion;
a second front portion connected to the second arm portion;
a back portion extending between the collar portion, the first arm portion, the second arm portion, the first front portion, and the second front portion; and
a collar pull tab in form of a strap, the collar pull tab being directly connected to the first front portion and extending outward from the first front portion and the collar pull tab [symbol] - [symbol] _having a pull fastener disposed on a distal end of the collar pull tab relative to where the collar pull tab is directly connected to the first front portion;
a pull mating fastener disposed on at least one of: the collar portion and the back portion;
a first wrist pull tab, in the form of a wrist strap that is directly connected to the first wrist portion and extending outward from the first wrist portion of the first arm portion, the first wrist pull tab having a first wrist tab fastener disposed thereon;
a first wrist tab mating fastener disposed on at least one of the first wrist portion and the first arm portion; and
wherein engaging the first arm fasteners and the first arm mating fasteners and engaging the first wrist tab fastener and the first wrist tab mating fastener is configured to secure the sweater around an arm of the wearer to supplement the existing attire of the wearer without disruption of pre-existing medical attachments connected to the wearer, and wherein engaging the pull fastener with the pull mating fastener is configured to wrap the pull fastener up and around the first arm portion and is configured to secure the sweater around the wearer.

2. The infusion friendly sweater of claim 1 further comprising:
a first front placket of the first front portion, the first front placket having a plurality of front fasteners disposed therein;
a second front placket of the second front portion, the second front placket having a plurality of front mating fasteners disposed therein;
wherein engaging the plurality of front fasteners with the plurality of front mating fasteners is configured to secure the sweater around the wearer without disruption of pre-existing medical device attachments.

3. The infusion friendly sweater of claim 1, wherein the first arm portion is composed_of a heat-encapsulating material.

4. The infusion friendly sweater of claim 1, wherein when the wearer keeps on existing attire prior to donning the infusion friendly sweater, the sweater is configured such that the first arm portion, the first front portion, the second front portion, the second arm portion, and the back portion have dimensions configured for draping over the wearer.

5. (canceled)

6. (canceled)

7. The infusion friendly sweater of claim 1 wherein the plurality of first arm fasteners and first arm mating fasteners include at least one of the following: magnets, buttons, and hook-and-loop fasteners.

8. The infusion friendly sweater of claim 1 further comprising:
a pocket accessible by the wearer upon donning the infusion friendly sweater, wherein the pocket is at least one of: an interior pocket and an exterior pocket.

9. (canceled)

10. An infusion friendly sweater configured to supplement existing attire of a wearer and configured to accommodate pre-existing medical attachments connected to the wearer, the sweater comprising:
a first arm portion having a first arm top placket and a first arm bottom placket, both the first arm top placket and the first arm bottom placket extending lengthwise from a collar portion to a first wrist portion of the first arm portion, the first arm top placket including a plurality of first arm fasteners and the first arm bottom placket including a plurality of first arm mating fasteners for engagement with the plurality of first arm fasteners;
a first front portion connected to the first arm portion,
a second arm portion having a second arm top placket and a second arm bottom placket, both the second arm top placket and the second arm bottom placket extending lengthwise from the collar portion to a second wrist portion of the second arm portion, the second arm top placket including a plurality of second arm fasteners and the second arm bottom placket including a plurality of second arm mating fasteners for engagement with the plurality of second arm fasteners;
a second front portion connected to the second arm portion;
a back portion extending between the collar portion, the first arm portion, the second arm portion, the first front portion, and the second front portion;
a collar pull tab in the form of a strap, the collar tab being directly connected to the first front portion and extending outward from the first front portion and the collar pull tab having a pull fastener disposed on a distal end of the collar pull tab relative to where the collar pull tab is directly connected to the first front portion;
a pull mating fastener disposed on at least one of: the collar portion and the back portion;
a first wrist pull tab, in the form of a wrist strap that is directly connected to the first wrist portion and extending outward from the first wrist portion of the first arm portion, the first wrist pull tab having a first wrist tab fastener disposed thereon;
a first wrist tab mating fastener disposed on at least one of the first wrist portion and the first arm portion; and
wherein engaging the first arm fasteners and the first arm mating fasteners and engaging the second arm fasteners with the second arm mating fasteners and engaging the first wrist tab fastener and the first wrist tab mating fastener is configured to secure the sweater around an arm of the wearer to supplement the existing attire of the wearer without disruption of pre-existing medical attachments connected to the wearer and engaging the pull fastener with the pull mating fastener is configured to wrap the full fastener up and around the first arm portion and is configured to secure the sweater around the wearer.

11. The infusion friendly sweater of claim 10, wherein the first arm portion is composed_of a heat-encapsulating material.

12. The infusion friendly sweater of claim 10, wherein when the wearer keeps on existing attire prior to donning the infusion friendly sweater, the sweater is configured such that the first arm portion, the first front portion, the second front portion, the second arm portion, and the back portion have dimensions configured for draping over the wearer.

13. (canceled)

14. (canceled)

15. The infusion friendly sweater of claim 10 wherein the plurality of first arm fasteners and first arm mating fasteners include at least one of the following: magnets, buttons, and hook-and-loop fasteners.

16. The infusion friendly sweater of claim 10 further comprising:
a pocket accessible by the wearer upon donning the infusion friendly sweater, wherein the pocket is at least one of: an interior pocket and an exterior pocket.

17. (canceled)

18. An infusion friendly sweater configured to supplement existing attire of a wearer and configured to accommodate pre-existing medical attachments connected to the wearer, the sweater comprising:
a first arm portion having a first arm top placket and a first arm bottom placket, both the first arm top placket and the first arm bottom placket extending lengthwise from a collar portion to a first wrist portion of the first arm portion, the first arm top placket including a plurality of first arm fasteners and the first arm bottom placket including a plurality of first arm mating fasteners for engagement with the plurality of first arm fasteners;
a first front portion connected to the first arm portion, the first front portion having a first front placket with a plurality of front fasteners disposed therein;
a second arm portion having a second arm top placket and a second arm bottom placket, both the second arm top placket and the second arm bottom placket extending lengthwise from the collar portion to a second wrist portion of the second arm portion, the second arm top placket including a plurality of second arm fasteners and the second arm bottom placket including a plurality of second arm mating fasteners for engagement with the plurality of second arm fasteners;
a second front portion connected to the second arm portion, the second front portion having a second front placket with a plurality of front mating fasteners disposed therein;
a back portion extending between the collar portion, the first arm portion, the second arm portion, the first front portion, and the second front portion; and
a collar pull tab in the form of a strap, the collar pull tab being directly connected to the first front portion and extending outward from the first front portion, the collar pull tab [symbol] - [symbol] _having a pull fastener disposed on a distal end of the collar pull tab relative to where the collar pull tab is directly connected to the first front portion;
a pull mating fastener disposed on at least one of: the collar portion and the back portion;
a first wrist pull tab, in the form of a wrist strap that is directly connected to the first wrist portion and extending outward from the first wrist portion of the first arm portion, the first wrist pull tab having a first wrist tab fastener disposed thereon;
a first wrist tab mating fastener disposed on at least one of the first wrist portion and the first arm portion; and
wherein engaging the first arm fasteners and the first arm mating fasteners, the second arm fasteners with the second arm mating fasteners, engaging the first wrist tab fastener and the first wrist tab mating fastener, and engaging the plurality of front fasteners with the plurality of front mating fasteners is configured to secure the sweater around the wearer and is configured to supplement the existing attire of the wearer without disruption of pre-existing medical attachments connected to the wearer and engaging the pull fastener with the pull mating fastener is configured to wrap the pull fastener up and around the first arm portion and is configured to secure the sweater around the wearer.

19. (canceled)

20. (canceled)
